(19) United States
(12) Patent Application Publication
(10) Pub. No.: US 2007/0026005 A1
(43) Pub. Date: Feb. 1, 2007

(54) Vaccine composition comprising il-12 adjuvant encapsulated in controlled-release microsphere

(76) Inventors: Young Chul Sung, Gyeongsangbuk-do (KR); Su Hyung Park, Seoul (KR); Jun Chang, Gyeongsangbuk-do (KR); Jong Moon Son, Incheon (KR); Sung Hee Lee, Seoul (KR); Won Bae Kim, Seoul (KR); Byong Moon Kim, Seoul (KR)

Correspondence Address: LUCAS & MERCANTI, LLP, 475 PARK AVENUE SOUTH, 15TH FLOOR, NEW YORK, NY, 10016, US

(21) Appl. No.: 10/571,196

(22) PCT Filed: Sep. 9, 2004
(86) PCT No.: PCT/KR04/02306

§ 371 Date: Oct. 2, 2006

(30) Foreign Application Priority Data
Sep. 9, 2003 (KR) 10-2003-0063343

(51) Int. Cl. A61K 39/00

(52) U.S. Cl. 424/184.1; 424/457

(57) ABSTRACT

Disclosed is a vaccine composition including a pathogenic antigen and IL-12 encapsulated in controlled release microspheres. Also, the present invention discloses a method of enhancing the adjuvant effect of IL-12 by employing an IL-12 adjuvant encapsulated in controlled release microspheres. IL-12, used as an adjuvant for a co-administered vaccine antigen in the vaccine composition, is released in vivo for a prolonged period of time by being encapsulated in controlled release microspheres, thereby maximizing its adjuvant effect.

### TECHNICAL FIELD

The present invention relates to a vaccine composition comprising a pathogenic antigen and an IL-12 adjuvant encapsulated in controlled release microspheres. Also, the present invention is concerned with a method of enhancing an adjuvant effect of IL-12 by employing an IL-12 adjuvant encapsulated in controlled release microspheres.

### BACKGROUND ART

The immune system uses various defense mechanisms for attacking pathogens, but not all of these mechanisms are activated after immunization. Protective immunity induced by vaccination is dependent on the capacity of a vaccine to elicit an appropriate immune response to resist, control or eliminate a pathogen. Depending on the pathogen, this requires a cellular (cell-mediated) or humoral immune response, which is determined by the nature of the T cells that was activated after immunization. For example, many bacterial, protozoal and intracellular parasitic and viral infections appear to require a strong cellular immune response for protection, while other pathogens, such as helminths, primarily respond to a humoral response.

Adjuvants are substances that enhance immune responses toward foreign antigens including pathogenic organisms. Suitable adjuvants include substances that do not serve as antigens in hosts but enhance immunity by increasing the activity of cells of the immune system. Adjuvants have been reported to function in various ways, including by increasing the surface area of an antigen, prolonging the retention of an antigen in the body to allow time for the lymphoid system to access the antigen, slowing the release of an antigen, targeting an antigen to macrophages, activating macrophages, and eliciting non-specific activation of the cells of the immune system (H. S. Warren et al., Annu. Rev. Immunol., 4:369 (1986).

Typical adjutants include water and oil emulsions, for example, Freund's adjuvant, and chemical compounds such as aluminum hydroxide or alum. At present, alum is the only practically used adjuvant. When alum is administered to the body in a form being bound to a protein, it is able to induce sustained release of the protein. However, in this case, alum itself coverts antigen-specific immune responses to Th2-type immune responses. Since, typically, Th1 responses, rather than Th2, responses are effective in inducing preventive immunity to pathogenic antigens, alum has limited application.

Current studies have been directed to the development of a method of delivering an antigen together with a cytokine involved in the induction of immune responses to achieve an immune-enhancing effect. Adjuvants belonging to this category include interleukins such as cytokines, for example, IL-1 or IL-12. In addition, adjuvants that do not follow mechanisms of interleukins but belong to this category include interferons, especially gamma-interferon and alpha-interferon, tumor necrosis factor (TNF) and granulocyte macrophage colony stimulating factor (GM-CSF).

When injected into the body in protein forms, the aforementioned cytokines have problems of being easily removed from the body due to their short half-lives and instability. According to previous studies, the persistence of cytokines is essential in effectively inducing antigen-specific immune responses (Sanjay Gurunathan et al., Nature Medicine 1998, 4:1409-1415). Thus, there is an urgent need for the development of methods capable of overcoming the problems and thus allowing effective vaccine development.

### DISCLOSURE OF THE INVENTION

Leading to the present invention, the intensive and thorough research into the effect of IL-12 on vaccination when used as an adjuvant in a vaccine composition in the form of being encapsulated in microspheres capable of achieving slow and sustained release of IL-12 in vivo, conducted by the present inventors, resulted in the finding that IL-12 encapsulated in microspheres remarkably increases immune responses to a vaccine for a prolonged period of time even in small amounts in comparison with a non-encapsulated protein form or a DNA form of IL-12.

Therefore, the present invention aims to maximize the adjuvant effect of IL-12 by employing IL-12 encapsulated in controlled release microspheres as an adjuvant in a vaccine composition.

The present invention relates to a vaccine composition for enhancing the adjuvant effect of IL-12 comprising a pathogenic antigen and an IL-12 adjuvant encapsulated in controlled release microspheres.

In addition, the present invention relates to a method of enhancing the adjuvant effect of IL-12, which is based on employing, as an adjuvant, an IL-12 adjuvant encapsulated in controlled release microspheres in a vaccine composition comprising a pathogenic antigen.

### BRIEF DESCRIPTION OF THE DRAWINGS

The above and other objects, features and other advantages of the present invention will be more clearly understood from the following detailed description taken in conjunction with the accompanying drawings, in which:

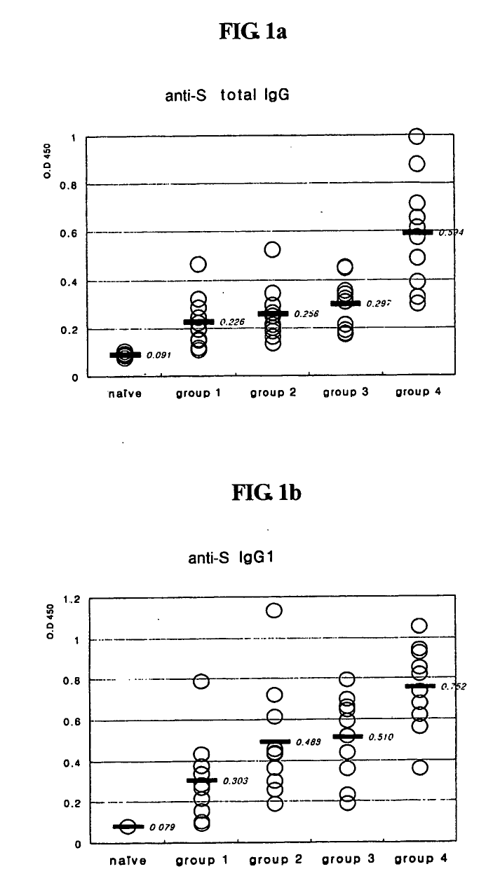

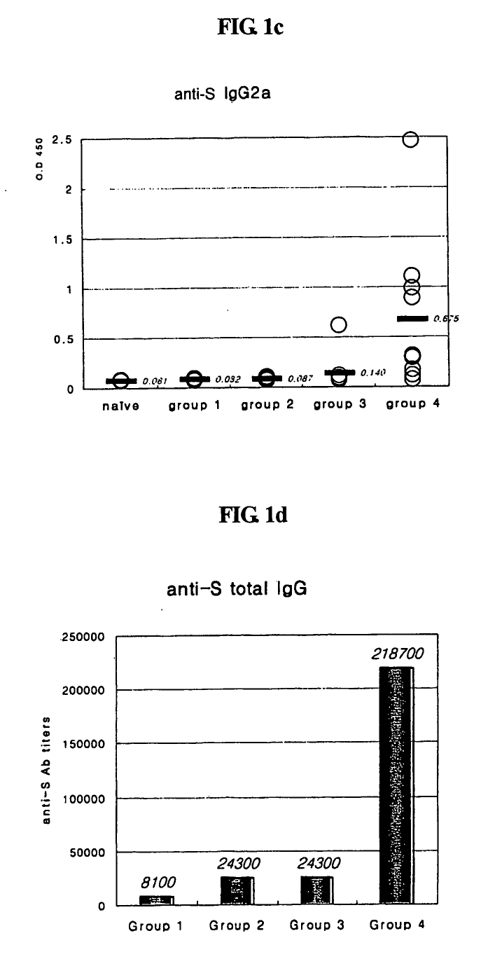

FIGS. 1a to 1f are graphs showing the antibody responses in mice subcutaneously immunized with a hepatitis B virus surface antigen, HBsAg, and rIL-12-encapsulating microspheres, wherein the titers of total serum IgG, IgG1, and IgG2a antibodies were measured by an anti-S ELISA, and each group was immunized with the following composition:
Group 1: HBsAg (0.5 μg)
Group 2: HBsAg (0.5 μg)+mock microspheres
Group 3: HBsAg (0.5 μg)+mock microspheres+rIL-12 (0.1 μg)
Group 4: HBsAg (0.5 μg)+rIL-12-encapsulating microspheres (0.1 μg);

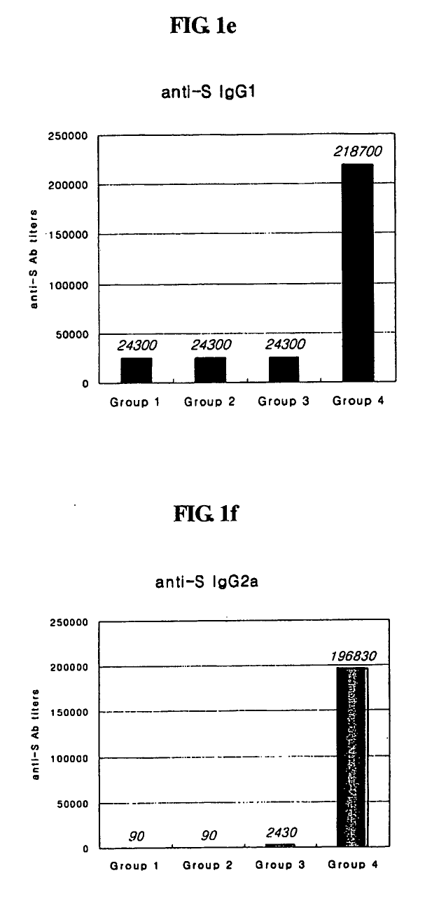

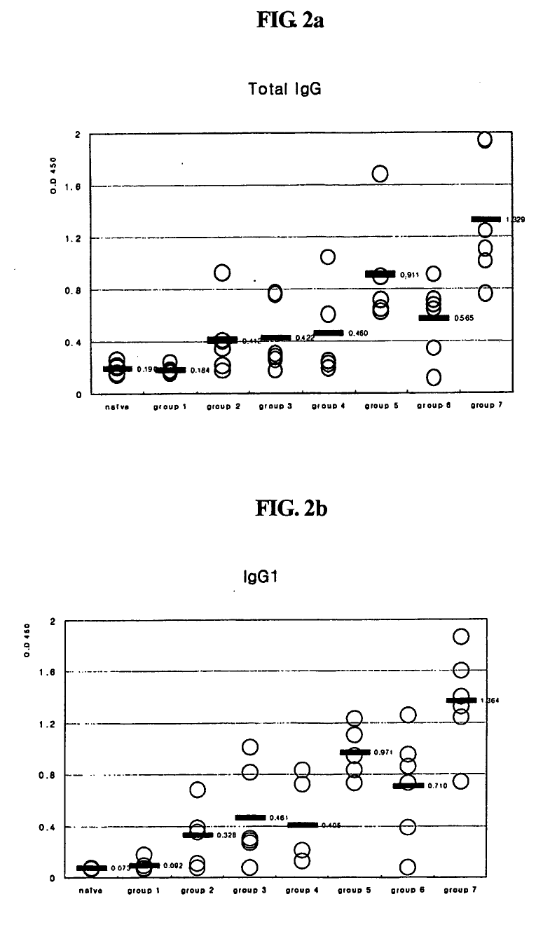

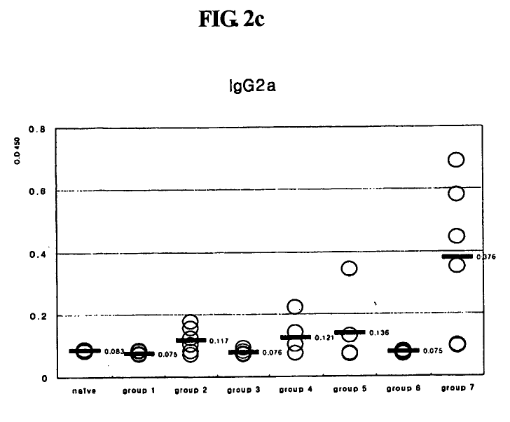

FIGS. 2a to 2c are graphs showing the adjuvant effect of rIL-12-encapsulating microspheres in mice immunized with various amounts of an antigen, wherein the adjuvant effect of the microspheres was analyzed by anti-S ELISA, and each group was immunized with the following composition:
Group 1: HBsAg (0.1 μg)
Group 2: HBsAg (0.1 μg)+rIL-12-encapsulating microspheres (0.1 μg)
Group 3: HBsAg (0.5 μg)
Group 4: HBsAg (0.5 μg)+rIL-12 (0.1 μg)
Group 5: HBsAg (0.5 μg)+rIL-12-encapsulating microspheres (0.1 μg)
Group 6: HBsAg (2.5 μg)
Group 7: HBsAg (2.5 μg)+rIL-12-encapsulating microspheres (0.1 μg);

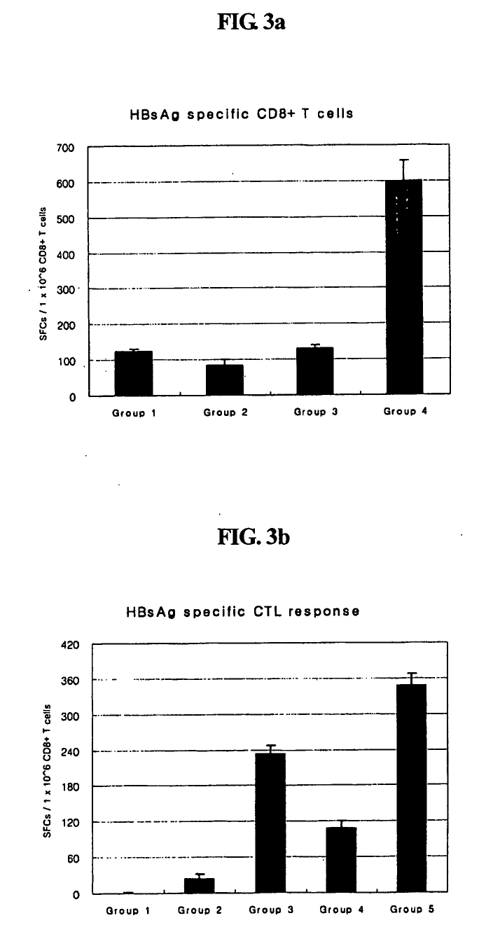

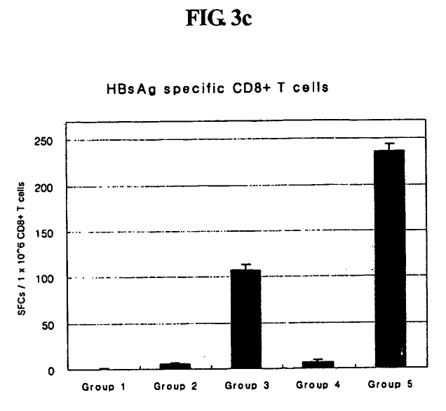

FIGS. 3a to 3c are graphs showing the results of an IFN-γ ELISPOT assay of CD8⁺ T cells stimulated with an HBV S-specific CTL epitope (IPQSLDSWWTSL), which were isolated from mice subcutaneously immunized with HBsAg and rIL-12-encapsulating microspheres, wherein each group in FIG. 3a was immunized with the following composition:
Group 1: HBsAg (0.5 μg)
Group 2: HBsAg (0.5 μg)+mock microspheres
Group 3: HBsAg (0.5 μg)+mock microspheres+rIL-12 (0.1 μg.)
Group 4: HBsAg (0.5 μg)+rIL-12-encapsulating microsphere (0.1 μg), and

each group in FIGS. 3b and 3c was immunized with the following composition:
Group 1: HBsAg (0.5 μg)
Group 2: HBsAg (0.5 μg)+rIL-12 (0.1 μg)
Group 3: HBsAg (0.5 μg)+rIL-12-encapsulating microspheres (0.1 μg)
Group 4: HBsAg (2.5 μg)
Group 5: HBsAg (2.5 μg)+rIL-12-encapsulating microspheres (0.1 μg);

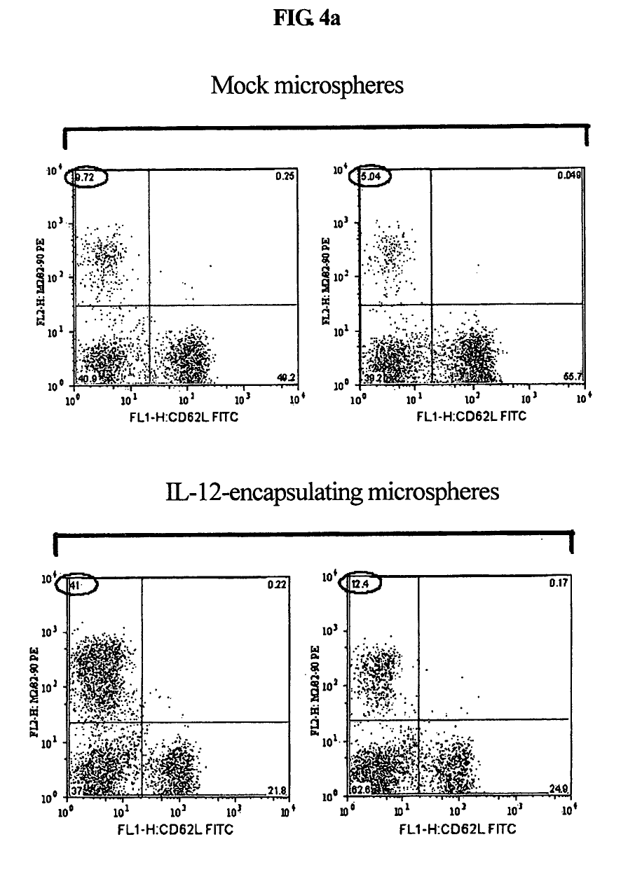

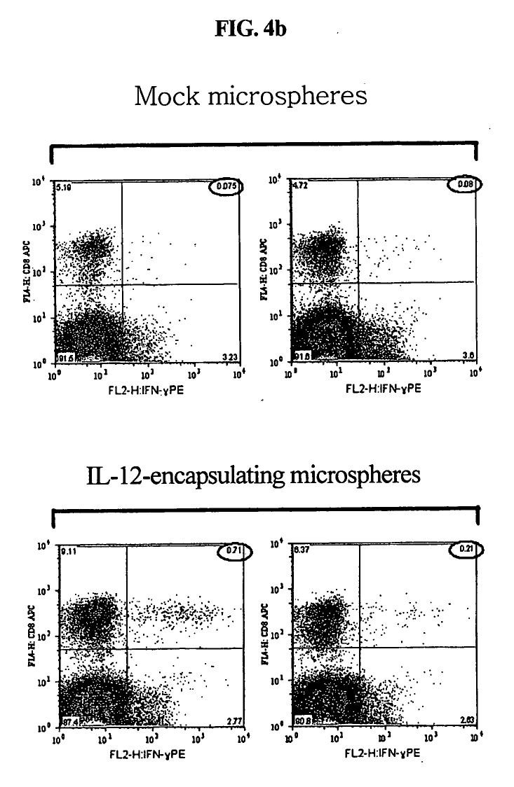

FIGS. 4a and 4b show the results of intracellular staining using FACS to determine the adjuvant effect of rIL-12-encapsulating microspheres, wherein mice were immunized intranasally twice at intervals of 2 weeks with M2/82-90 peptide, known as a respiratory syncytial virus-specific CTL epitope, and rIL-12-encapsulating microspheres, and each group was immunized with the following composition:
Group 1: M2/82-90 (20 μg)+mock microspheres
Group 2: M2/82-90 (20 μg)+rIL-12-encapsulating microspheres (0.1 μg);

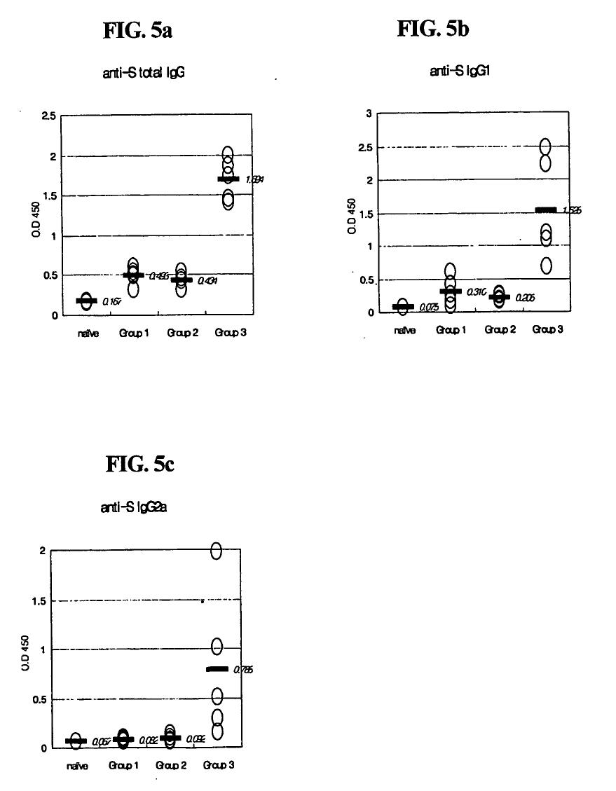

FIGS. 5a and 5b are graphs showing the antibody responses of mice immunized with HBsAg and rIL-12-encapsulating microspheres to compare IL-12 DNA and IL-12 protein encapsulated in microspheres for adjuvant effects, wherein the titers of total serum IgG, IgG1, and IgG2a antibodies were measured by an anti-S ELISA, IL-12 DNA was intramuscularly administered, HBsAg and IL-12 protein-encapsulating microspheres were subcutaneously administered, and each group was immunized with the following composition:
Group 1: HBsAg (0.5 μg)
Group 2: HBsAg (0.5 μg)+IL-12 DNA vaccine (10 μg)
Group 3: HBsAg (0.5 μg)+rIL-12-encapsulating microsphere (0.1 μg);

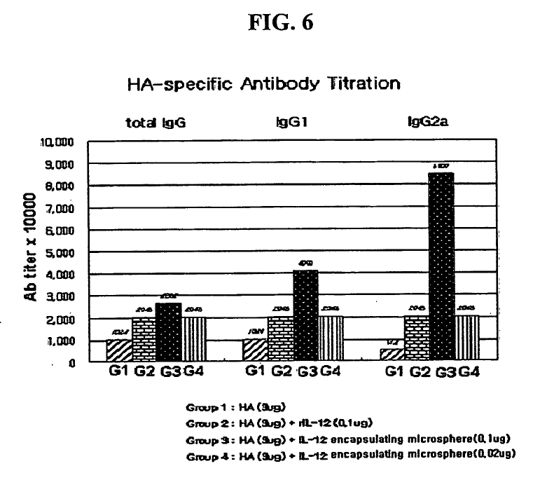

FIG. 6 is a graph showing the antibody responses of mice intranasally immunized with an influenza virus surface antigen, influenza HA, and rIL-12-encapsulating microspheres, wherein the titers of total serum IgG, IgG1, and IgG2a antibodies were measured by an anti-S ELISA, and each group was immunized with the following composition:
Group 1: HA (3 μg)
Group 2: HA (3 μg)+rIL-12 (0.1 μg)
Group 3: HA (3 μg)+rIL-12-encapsulating microspheres (0.1 μg)
Group 4: HA (3 μg)+rIL-12-encapsulating microspheres (0.02 μg);

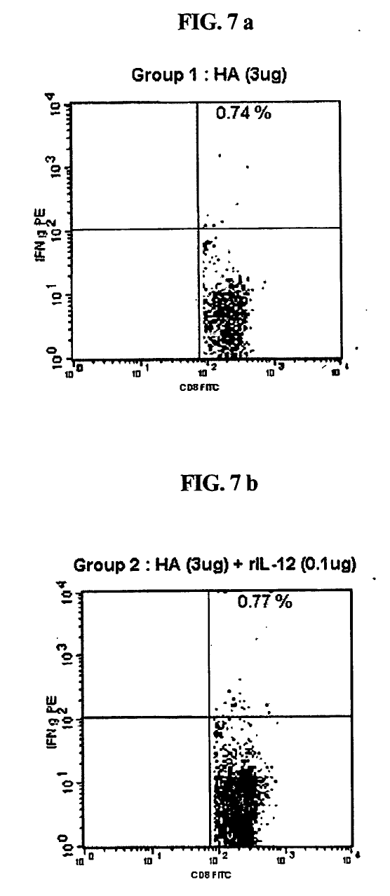

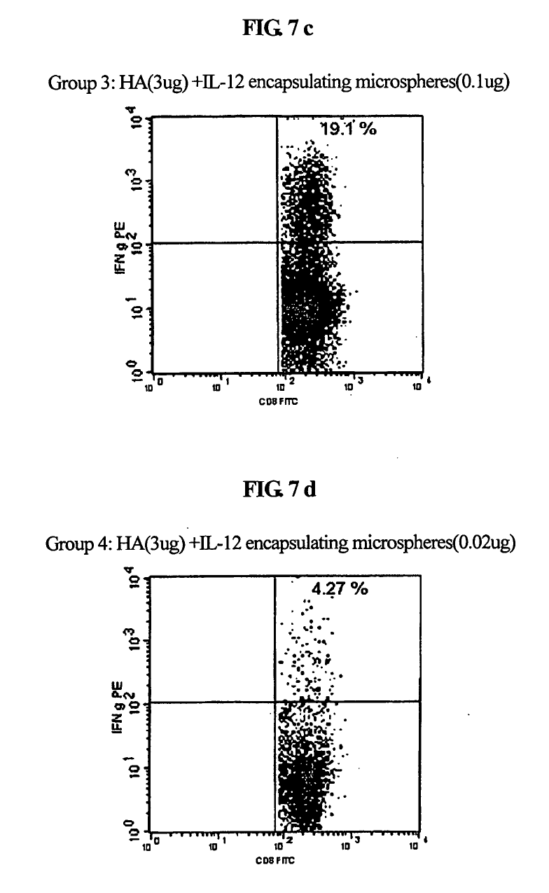

FIGS. 7a to 7d are graphs showing the results of intracellular staining using FACs of CD8₊ T cells stimulated with an HA-specific CTL epitope, which were isolated from the mouse lung tissue at five days after influenza infection. The mice were intranasally immunized with an influenza virus surface antigen, HA protein, and rIL-12-encapsulating microspheres, and each mice was challenged with lethal doses of influenza virus at 9 weeks after last immunization. Each group was immunized with the following composition:
Group 1: HA (3 μg)
Group 2: HA (3 μg)+rIL-12 (0.1 μg)
Group 3: HA (3 μg)+rIL-12-encapsulating microspheres (0.1 μg)
Group 4: HA (3 μg) +rIL-12-encapsulating microspheres (0.02 μg); and

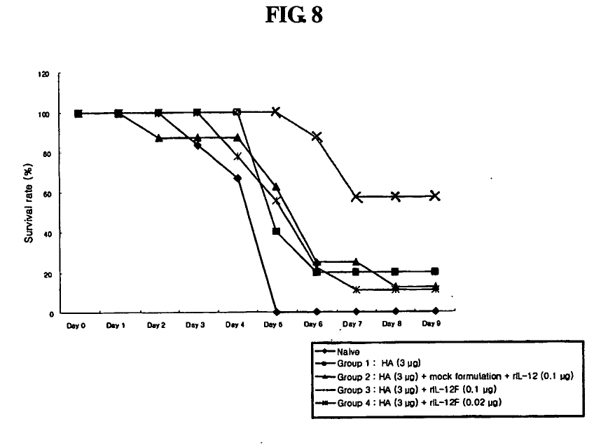

FIG. 8 is a graph showing the survival rate of mice which were intranasally challenged with an influenza virus surface antigen, HA protein, and rIL-12-encapsulating microspheres and were infected with lethal doses of influenza virus by an intranasal route, wherein each group was immunized with the following composition:
Group 1: HA (3 μg)
Group 2: HA (3 μg)+rIL-12 (0.1 μg)
Group 3: HA (3 μg)+rIL-12-encapsulating microspheres (0.1 μg)
Group 4: HA (3 μg)+rIL-12-encapsulating microspheres (0.02 μg).

### BEST MODE FOR CARRYING OUT THE INVENTION

In one aspect, the present invention provides a vaccine composition for enhancing the adjuvant effect of IL-12 comprising a pathogenic antigen and an IL-12 adjuvant encapsulated in controlled release microspheres.

The term “pathogenic antigen”, as used herein, refers to an antigen that is derived from a pathogenic microorganism to which a host induces an immune response. The pathogenic microorganism may include an intracellular parasite, such as a virus, bacterium or protozoan, and an extracellular parasite, such as a helminth or bacterium.

The pathogenic antigen from a pathogenic microorganism includes proteins or fragments thereof (e.g., protein degradation products), peptides (e.g., synthetic peptides, polypeptides), glycoproteins, carbohydrates (e.g., polysaccharides), lipids, glycolipids, hapten conjugates, whole organisms (killed or attenuated organisms) or portions thereof, toxins and toxoids.

In addition, the pathogenic antigen may be a DNA sequence encoding an antigen from a pathogenic microorganism. This DNA sequence, together with a suitable promoter sequence, may be directly used as an antigen administered with a cytokine adjuvant. Alternatively, the DNA sequence may be introduced into other vaccine strains of the pathogenic microorganism, and, upon expression in vivo, may provide an antigen.

The pathogenic antigen may be obtained or induced from a variety of pathogens or organisms. For example, the pathogenic antigen may be obtained or induced from bacteria (e.g., Salmonella dublin, Borrelia burgdorferi, Bacillus, treptococcus, Bordetella, Listeria, Bacillus anthracis, Streptococcus pneumoniae, Neiseria meningiditis, H. influenza, etc.); viruses (e.g., hepatitis B virus, hepatitis C virus, acute respiratory virus, measles virus, poliovirus, human immunodeficiency virus, influenza virus, parainfluenza virus, respiratory syncytial virus, herpes simplex virus, Ebola virus, lymphocytic choriomeningitis virus, murine retrovirus, Rabies virus, Smallpox virus, adenovirus, Varicella-zoster virus, enterovirus, rotavirus, yellow fever virus, etc.); mycobacteria (e.g., Mycobacterium tuberculosis, etc.); parasites (e.g., Leishmania, Schistosomes, Tranpanosomes, toxoplasma, pneumocystis, etc.); and fungi (e.g., Histoplasma, Candida, Cryptococcus, Coccidiodes, Aspergillus, etc.), but the present invention is not limited to these examples.

Preferably, the pathogenic antigen contained in the vaccine composition of the present invention may be obtained or induced from viruses. For example, the pathogenic antigen may be derived from a broad range of viruses including hepatitis viruses, acute respiratory virus, measles virus, poliovirus, human immunodeficiency virus, influenza virus, parainfluenza virus and respiratory syncytial virus.

In particular, in the case of viruses causing chronic diseases or having high mutation rates, such as hepatitis B virus, hepatitis C virus, human immunodeficiency virus and influenza virus, Th1-type T cell immune responses are known to be more important in inducing preventive immunity or eliminating viruses than antibody immune responses, and IL-12 is known to be essential for eliciting such immune responses. Also, in the case of bacteria such as Mycobacterium tuberculosis, elevation of T cell immune responses by IL-12 is known to be critical in inducing preventive immunity. Thus, the pathogenic antigen contained in the vaccine composition of the present invention is preferably derived from hepatitis B virus, hepatitis C virus, human immunodeficiency virus, influenza virus or Mycobacterium.

The pathogenic antigen contained in the vaccine composition of the present invention may be obtained using techniques known in the art. For example, the antigen may be directly isolated (purified) from a pathogen, induced using a chemical synthetic method, or using a recombinant DNA method. Also, the antigen may be obtained from commercially available products. The antigen useful in the present invention includes one or more B and/or T cell epitopes (e.g., T helper cell or cytotoxic T cell epitopes), and may be easily determined by those skilled in the art.

Preferably, the vaccine composition of the present invention may include a pathogenic antigen in a protein or peptide form. Preferably, a protein or peptide form of the pathogenic antigen may be directly isolated, chemically synthesized or prepared by a recombinant DNA technique, and more preferably by the recombinant DNA technique.

If desired, the pathogenic antigen contained in the vaccine composition of the present invention, as described above, may be contained in a dispersion system to achieve its sustained release, which is selected from the group consisting of macromolecular complexes, nanocapsules, microspheres, beads, oil-in-water emulsions, micelles, mixed micelles, liposomes and resealed erythrocytes.

Interleukin-12 (IL-12), contained in the vaccine composition of the present invention as an adjuvant, is known to be a major element in enhancing the efficacy of a vaccine when cellular immunity is required.

IL-12 is secreted by antigen presenting cells (APC) including macrophages and monocytes after appropriate stimulation, and functions to modulate various immune responses in vivo. In detail, IL-12 has a broad range of biological activities including the differentiation of T helper 1 (Th1) cells and natural killer (NK) cells, the regulation of production of various cytokines, the enhancement of immune responses mediated by Th1 cells, the differentiation of CD8⁺ T cells and the proliferation of hematopoietic cells (Hsieh, C. S., et al., Science, 260:547-549, 1993). In particular, IL-12 plays a critical role in regulating immune responses by improving the hydrolysis capacity of CTL cells (cytotoxic T lymphocytes) and NK cells (Robertson, M. J., and J. Ritz., Oncologist, 1:88-97, 1999; Trinchieri, G., Annu. Rev. Immunol., 13:251-276, 1995). According to other reports, synthesis of biologically active IL-12 decreases by about five times in AIDS patients (Chehimi, J. et al., J. Exp. Med., 179:1361-1366, 1994), and immunity against mycobacteria greatly decreases in IL-12 receptor-deficient patients (de Jong R. et al., Science, 280:1435-1438, 1998). Since IL-12, by virtue of these biological activities, can induce potent in vivo immune responses against viruses, bacteria or various cancers in early stages, it is increasingly used for developing various therapeutic agents.

The potential use of IL-12 as an effective vaccine or therapeutic agent for various diseases requiring cellular immune responses, as mentioned above, is also based on the hypothesis that IL-12 participates in the proliferation of memory Th1 cells and memory CTL (Stobie, L. et al., Proc. Natl. Acad. Sci. USA, 97:8427-8432, 2000; Mortarini, R. et al., Cancer Res., 60:3559-3568, 2000; Mbawuike, I. N. et al., J. Infect. Dis., 180:1477-1486, 1999). In particular, with respect to the most severe problems, metastasis and recurrence, upon treatment of various tumors, the induction of memory immune responses is essential. However, to date, an accurate mechanism explaining these effects of IL-12 has not been known. Some recent reports suggest that, since increased levels of IFN-γ during Th1 cell differentiation by IL-12 has an antiproliferative effect, IL-12 may induce memory immune responses by suppressing apoptosis of CD4⁺T cells (Fuss, I. J. et al., Gastroenterology 117:1078-1088, 1999; Marth, T. et al., J. Immunol. 162:7233-7240, 1999). Also, another hypothesis involving IL-12 inducing memory 5 immune responses has been suggested, based on the notion that elevated levels of IFN-γ by IL-12 promote expression of IL-15 participating in potent and selective stimulation of memory CD8⁺ T cells (Zhang, X. et al., Immunity 8:591-599, 1998). These reports suggest that IL-12 may participate in both primary immune responses and memory immune responses. Thus, IL-12 has a potential to be particularly valuably used in vaccine immunization.

IL-12 as an adjuvant has been reported not to induce the uncontrolled production of other cytokines, not to induce any sensitization in the case of originating from humans and to have no obvious side effects upon subcutaneous injection.

When IL-12 is administered in a DNA form, its endogeneous expression is induced, and the expression of IL-12 lasts for a longer period of time than the case of being administered in a protein form. Based on this fact, Sanjay Gurunathan et al. stated in Nature Medicine 4:1409-1415, 1988 that the administration of an antigenic protein in combination with IL-12 DNA induces more long-lasting immune responses against intracellular infections such as Leishmania major and Mycobacterium tuberculosis.

Unlike these reports, the present inventors found that, when a protein form of IL-12 used as an adjuvant is encapsulated in sustained release microspheres and used in a vaccine composition, it sustains and remarkably enhances antibody and cellular immune responses to a vaccine even in small amounts for a longer period of time than a DNA form of IL-12.

In detail, the present inventors subcutaneously administered IL-12 encapsulated in microspheres to mice in combination with a HBV preventive vaccine, recombinant HBsAg. This combination resulted in total IgG and IgG1 antibody responses 10 to 30-fold higher than HBsAg alone, HBsAg plus native form of IL-12 not encapsulated in microspheres and HBsAg plus IL-12 DNA. In particular, IgG2a antibody responses, as an indicator for Th1 immune responses, were found to remarkably increase by 80 to 2000 times by the IL-12 encapsulated in microspheres. CTL immune responses were also found to increase about 6 times by the IL-12 encapsulated in microspheres. In addition, when the IL-12 encapsulated in microspheres was intranasally administered in combination with an M2/82-90 peptide of RSV, CTL responses were 5 to 10-fold elevated. Further, in an influenza HA vaccine model, the use of the IL-12-encapsulating microspheres induced 2 to 3-fold increased antibody responses and 4 to 25-fold increased CTL responses against a co-administered vaccine. These results indicate that the IL-12-encapsulating microspheres are applicable to various vaccines to enhance immune responses against the vaccines.

Thus, the IL-12, encapsulated in sustained release microspheres, contained in the vaccine composition of the present invention indicates its protein form.

In comparison with a DNA form of IL-12, a protein form of IL-12, contained in the present vaccine composition as an adjuvant, has the following advantages. Protein forms of cytokines are typically administered to the body via the subcutaneous route, but subcutaneous injection of cytokines in DNA forms is known to lead to unsatisfactory effects. In this regard, when a vaccine in a protein form is administered subcutaneously while a DNA form of IL-12 as an adjuvant is administered intramuscularly, the vaccine antigen and the adjuvant do not exist simultaneously in an identical region, thereby making it difficult to attain desired effects. In addition, IL-12 should be present in the early phase of the antigen presentation to be served as an adjuvant for a co-administered vaccine. However, when the immunization is carried out by intramuscularly administering IL-12 DNA, it takes much time for IL-12 DNA to express in the body (generally muscular cells) and move to a desired site. In particular, the use of an IL-12 protein in a form of being encapsulated in microspheres make it possible to control the in vivo release duration by varying the composition of the microspheres. In contrast, in the case of using IL-12 DNA, IL-12 DNA expresses in very low levels, the persistence of expressed IL-12 is not controlled, and clinical safety is not ensured, thereby requiring further studies.

The term “IL-12”, as used herein, refers to an IL-12 protein, a subunit thereof, a multimer of the subunit, a functional fragment of IL-12, and a functional equivalent and/or isoform of IL-12. The functional fragment of IL-12 includes fragments that induce immune responses to an antigen when administered together with the antigen. In addition, the functional equivalent or isoform of IL-12 includes IL-12 variants that are altered to have biological activity similar to native IL-12, that is, modified IL-12 proteins having an ability to induce an immune response to an antigen when administered together with the antigen. In particular, this includes modified IL-12 proteins with an alteration of a specific amino acid residue, which are designed to have higher immunoenhancing activity.

IL-12 may be obtained from various origins or synthesized using a known technique. For example, IL-12 may be purified (isolated) from a native origin (e.g., mammals such as humans), produced by chemical synthesis, or produced by a recombinant DNA technique. In addition, IL-12 may be obtained from commercially available products. In particular, IL-12 may be preferably isolated, synthesized or produced by a recombinant DNA technique from a human origin.

IL-12 as an adjuvant may be used in an amount of about 1 ng to about 20 μg, and preferably about 100 ng to about 5 μg, but the present invention is not limited to this range.

A majority of proteins, when orally administered, lose their active structures under the acidic environment of the stomach, are destroyed by enzymatic degradation, and are absorbed in very low levels by the mucous membrane of the stomach and the intestinal. Thus, most protein drugs are administered parenterally, that is, by intravenous injection, subcutaneous injection or intramuscular injection. Even after administration via these routes, most protein drugs should be repeatedly injected due to their short half-lives. For controlled release of these proteins, these ingredients may be included in a dispersion system selected from the group consisting of macromolecular complexes, nanocapsules, microspheres, beads, oil-in-water emulsions, micelles, mixed micelles, liposomes and resealed erythrocytes.

The most commonly used biodegradable polymers for sustained injectable preparations of proteins are polyesters as synthetic polymers, which include polylactide (PLA), polyglycolide (PGA) and their copolymer, poly(lactide-co-glycolide) (PLGA). In addition to these synthetic polyesters, natural polymers are studied as matrices for sustained formulations of protein drugs, which include lipids such as lipids, fatty acids, waxes and their derivatives; proteins such as albumin, gelatin, collagen and fibrin; and polysaccharides such as alginic acid, chitin, chitosan, dextran, hyaluronic acid and starch. Non-limiting examples of the lipids include fatty acids (e.g., myristic acid, palmitic acid, stearic acid, etc.), monoacylglycerols (e.g., pamoic acid, glyceryl myristate, glyceryl palmitate, glyceryl stearate, etc.), sorbitan fatty acid esters (e.g., sorbitan myristate, sorbitan palmitate, sorbitan stearate, etc.), triglycerides (e.g., diacyl glycerol, trimyristin, tripalmitin, tristearin, etc.), phospholipids (e.g., phosphatidyl choline, phosphatidyl ethanolamine, phosphatidyl acid, phosphatidyl serine, phosphatidyl glycerol, phosphatidyl inositol, cardiolipin, etc.), sphingolipids (e.g., sphingosine, ceramide, sphinganine, etc.), waxes, and salts and derivatives thereof.

In particular, among the aforementioned biodegradable polymers, the polyesters, such as PLA, PGA or PLGA, are approved to be biocompatible and safe to the body because they are metabolized in vivo to harmless lactic acid and glycolic acid by hydrolysis. The degradation of the polyesters may be controlled at various rates according to the molecular weight, the ratio of the two monomers, the hydrophilicity, and the like, for various durations ranging from a short period of one to two weeks to a long period of one to two years. The polyesters are polymeric substances that have been approved for use in humans in several tens of countries, including by the U.S. Food and Drug Administration (FDA), and commercialized. Therefore, the polyesters may be preferably used in the present invention. In particular, the polyesters such as PLGA or PLA may be preferably used in the present invention.

To capture a protein into the aforementioned polymeric matrix, various methods may be used, including coacervation, spray drying-dependent encapsulation, and solvent evaporation in an organic or water phase. Among the above methods, W/O/W double emulsion-solvent evaporation has been widely used in manufacturing sustained release microparticles containing protein drugs because most protein drugs are water-soluble. In this W/O/W technique, a protein or water-soluble drug is dissolved in water, and this aqueous phase is dispersed in an organic phase containing a biodegradable polymer using an ultrasonicator or homogenizer, in order to give a primary emulsion. Again, this primary emulsion is dispersed in a secondary aqueous phase containing a surfactant such as polyvinylalcohol, so as to provide a secondary emulsion. As the organic solvent is removed from this system by heating or under pressure, the polymer is solidified to form microparticles. The microparticles are recovered by centrifugation or filtration and freeze-dried to give biodegradable microparticles containing the protein or water-soluble drug.

To minimize denaturation and irreversible coagulation of a protein when the protein is entrapped into a biodegradable polymer, a stabilizer may be used in an aqueous solution of the protein, which is exemplified by trihalose, mannitol, dextran and polyethylene glycol. These stabilizers form a hydrated layer around a protein and thus reduce the interaction between a protein and an organic solvent, thereby preventing the denaturation and irreversible coagulation of the protein to some extent. In addition, the protein denatruation may be minimized by directly dispersing in an organic solvent a protein drug in a powder form rather than in a form of being dissolved in an aqueous solution.

The term “sustained or controlled release”, as used herein, means that the vaccine composition of the present invention, containing an IL-12 adjuvant encapsulated in microspheres, requires an hour or longer to release a major portion of the active substance into the surrounding medium, for example, 24 hours or longer.

Microsphere-based drugs may be utilized for oral ingestion, implantation, or external application to the skin or a mucous membrane. Where implantation is desired, microspheres may be implanted subcutaneously, constitute a portion of a prosthesis, or be inserted into a cavity of the human body. Subcutaneous implantation using a syringe consists of injecting an implant directly into a subcutaneous tissue, and is a particularly effective method for controlled drug delivery. The IL-12-encapsulating microspheres according to the present invention may be suspended in a physiological buffer and introduced into a desired site using a syringe.

When applied to a desired site of the body by a desirable mode, the IL-12-encapsulating sustained release microspheres provides sustained release of IL-12 by allowing IL-12 to diffuse through the microspheres or by allowing the microspheres to degrade in vivo upon contact with body fluids. When the microspheres are degraded in a site where the microspheres are injected, the degree of their degradation, that is, the release rate of the active substance, may be regulated by the degree of crosslinking of the microspheres.

The IL-12-encapsulating microspheres may be about 20 nm to 50 μm in diameter. The microspheres of this sphere size may be suspended in a pharmaceutical buffer and introduced into a patient using a syringe.

The vaccine composition containing IL-12 encapsulated in microspheres according to the present invention may be administered to a patient, whether displaying a pathogenic state caused by a pathogen or not, so as to suppress or delay the incidence of a disease or alleviate or eliminate the disease.

The vaccine composition for prevention or therapy according to the present invention may be administered in an immunologically effective amount for prevention or therapy. The term “immunologically effective amount” means an amount suitable for inducing an immune response. A specific amount may vary depending on the patient's age and weight, the severity of illness and administration methods, and a suitable amount may be easily determined by those skilled in the art. The vaccine composition may be contained in a pharmaceutically or physiologically acceptable vehicle, for example, physiological or phosphate-buffered saline, or ethanol or polyols, such as glycerol or propylene glycol.

If desired, the vaccine composition of the present invention may further include additional adjuvants (e.g., vegetable oils or emulsions thereof), surfactants (e.g., hexadecylamine, octadecyl amino acid esters, octadecylamine, lisolecithin, dimethyldioctadecylammonium bromide, N,N-dioctadecyl-N′, N′-bis (2-hydroxyethylpropane diamine), methoxyhexadecylglycol, pluronic polyols), polyamines (e.g., pyrans, dextransulfate, poly IC, carbopol), peptides (e.g., dimethylglycine), immunostimulatory complexes, oil emulsions, lipopolysaccharides (e.g., d3-MPL (3-O-deacylated monophosphoryl lipid A; RIBI ImmunoChem Research, Inc., Hamilton, Mont.)), and inorganic gels.

The vaccine composition of the present invention may be administered by various routes, for example, parenterally, intraarterially, subcutaneously, transdermally, intramuscularly, intraperitoneally, intravenously, orally and intranasally.

A better understanding of the present invention may be obtained through the following examples which are set forth to illustrate, but are not to be construed as the limit of the present invention.

### EXAMPLE 1

### Preparation of rIL-12-Encapsulating Microspheres and Mock Microspheres

IL-12-encapsulating microspheres were prepared by a W/O/W double emulsion-solvent evaporation method.

A murine recombinant IL-12 protein (rIL-12) (R&D System) and bovine serum albumin (BSA) were added to PBS buffer according to the composition summarized in Table 1, below, so as to give a W1 solution (total volume: 500 μl). The W1 solution was emulsified in 1.2 ml of DCM (dichloromethane) (oil phase (O)) supplemented with a polymeric carrier PLGA (polylactide-co-glycolide) and an emulsifier Pluronic L121 using a homogenizer, thus providing a primary emulsion (W1/O). Again, the primary emulsion was emulsified in distilled water (W2) containing another emulsifier PVA (polyvinylalchol) using a homogenizer, thus providing a secondary emulsion (W1/O/W2). The secondary emulsion was solidified to form microspheres, filtered and dried.

TABLE 1

| W1 | | | Oil | | W2 |
|---|---|---|---|---|---|
| mlL-12 | BSA | Buffer | PLGA | CH₂Cl₂ | 1% PVA |
| 50 μg | 12.5 mg | 500 μg | 500 mg | 1.2 ml of | |
| | | | | 2% pluronic | |
| | | | | L121 | |

The rIL-12-encapsulating microspheres were analyzed using a laser scattering particle size distribution analyzer (Hydro-2000MU, MALVERN) for sphere size, an optical microscope (IX70, Olympus) and a SEM microscope (JSM 890, JEOL LTD) for morphology, and a size exclusion (SE)-HPLC column (TOSOH) and a Dc protein analyzer (Bio-Rad) for loading (%).

Mock microspheres as a negative control were prepared according to the same procedure as described above except for not using rIL-12.

### EXAMPLE 2

### Enhanced HBsAg-Specific Antibody Responses by the rIL-12-Encapsulating Microspheres

The adjuvant effect of the rIL-12-encapsulating microspheres with respect to antibody responses was investigated as follows. A hepatitis B virus surface antigen, HBsAg (Euvax B, LGCI Co. Ltd.) and the microspheres prepared in Example 1 were suspended in 100 μl of a suspension solution (3% carboxymethyl celluose, 8.7 mg/ml NaCl, 0.1% Tween 20). Five-week old BALB/c CrSlc mice were subcutaneously immunized with the resulting suspension. After four weeks, the titers of total serum IgG, IgG1, and IgG2a antibodies were measured by an anti-S ELISA to determine whether anti-HBsAg antibody responses had been induced. In FIGS. 1a, 1b, 1c, 2a, 2b and 2c, antibody responses were expressed as absorbance at 450 nm. FIGS. 1d, 1e and 1f show the results of quantitative comparison for antibody responses expressed as antibody titers measured by an end-point dilution assay.

As shown in FIG. 1a, the strongest total IgG antibody responses were observed in Group 4 administered with the rIL-12-encapsulating microspheres. As shown in FIG. 1d, the Group 4 was also found to produce about 9 to 27-fold stronger total IgG antibody responses than other groups. In contrast, in both Group 2 administered with mock microspheres and Group 3 administered with mock microspheres plus rIL-12 protein, no significant increase was observed (see, FIGS. 1a and 1d). Also, in the case of IgG1 responses, the Group 4 administered with the rIL-12-encapsulating microspheres was found to induce about 9-fold stronger immune responses (see, FIGS. 1b and 1e). In the case of IgG2a responses, only the Group 4 administered with the rIL-12-encapsulating microspheres induced very-strong significant antibody responses (see, FIG. 1c). As shown in FIG. 1f, the Group 4 was found to induce 81 to 2187-fold stronger IgG2a antibody responses than other groups.

These results indicate that the rIL-12-encapsulating microspheres enhance host's antibody and T-helper 1 immune responses to a co-administered antigen, and that the present microspheres designed to continuously release IL-12 greatly improve the adjuvant effect of IL-12.

Also, mice were immunized with different amounts of the antigen, and the adjuvant effect of the microspheres was evaluated by anti-S ELISA. As shown in FIGS. 2a to 2c, even when the antigen was used even in small amounts, the co-administration of the IL-12-encapsulating microspheres also was found to lead to strong antibody responses. These results indicate that the present microspheres have an excellent effect on adjuvantation of an antigen regardless of administered amounts of the antigen.

### EXAMPLE 3

### Enhanced HBsAg-Specific CTL Responses by the rIL-12-Encapsulating Microspheres

The adjuvant effect of the rIL-12-encapsulating microspheres with respect to CTL responses was investigated as follows. HBsAg (Euvax B, LGCI Co. Ltd.) and the microspheres were suspended in 100 μl of a suspension solution (3% carboxymethyl celluose, 8.7 mg/ml NaCl, 0.1% Tween 20). Five-week old BALB/c CrSlc mice were subcutaneously immunized with the resulting suspension. After 13 weeks (primary test) and after 9 and 24 weeks (secondary test), the spleen was excised from the immunized mice, and CD8⁺ T cells were isolated from the spleen by a magnetic bead cell separation technique (MACS). The isolated CD8⁺ T cells were subjected to an IFN-γ ELISPOT assay using HBV S-specific CTL epitope (IPQSLDSWWTSL) as a stimulus.

FIG. 3a shows the results 13 weeks after immunization. As shown in FIG. 3a, a group co-administered with the rIL-12-encapsulating microspheres displayed remarkably enhanced CTL responses in comparison with other groups. As shown in FIGS. 3b and 3c, like the results of antibody responses, this excellent effect of the rIL-12-encapsulating microspheres on enhancing CTL responses was found to be achieved regardless of the amount of the antigen used in the immunization. In addition, this enhancement of CTL responses by the rIL-12-encapsulating microspheres was maintained 24 weeks after immunization (see, FIG. 3c).

### EXAMPLE 4

### Enhanced RSV-Specific CTL Responses by the rIL-12 Encapsulating Microspheres

To determine whether the rIL-12-encapsulating microspheres have the vaccine adjuvanting effect on another antigen, a respiratory syncytial virus (RSV) was used as a vaccine antigen. In addition, the rIL-12-encapsulating microspheres were evaluated for their immunoenhancing effects upon the use of an antigen of a peptide type instead of a protein type and upon the intranasal administration of the microspheres instead of subcutaneous injection. First, an M2/82-90 peptide (Peptron Co. Ltd.), identified as a CD8⁺ T cell epitope, and the IL-12-encapsulating microspheres were suspended in 50 μl of a suspension solution (PBS). Five-week old BALB/c CrSlc mice were intranasally immunized twice at intervals of 2 weeks with the resulting suspension. After two weeks, lung lymphocytes were isolated from the immunized mice, and FACS was carried out to determine whether RSV M2/82-90 specific CTL responses are induced. FIG. 4a shows the results of quantitative analysis using FACS for the percentage of M2/82-90-specific CD8⁺ T cells among total lung CD8⁺ T cells. FIG. 4b shows the results of quantitative analysis using FACS of stained cells for the percentage of IFN-γ-positive M2/82-90-specific CTL. As shown in FIG. 4a, in comparison with a mock microsphere-administered group, in a rIL-12-encapsulating microsphere-administered group, M2/82-90-specific CD8+T cells were significantly increased. In addition, as shown in FIG. 4b, in the rIL-12-encapsulating microsphere-administered group, IFN-y-secreting M2/82-90-specific CTL was significantly increased in comparison-with the other group. These results indicate that the rIL-12-encapsulating microspheres are applicable not only to the subunit vaccine but also to the peptide vaccine and applicable various types of antigens regardless of the administration route of the microspheres.

### EXAMPLE 5

### Comparison of the rIL-12 Protein-Encapsulating Microspheres and IL-12 DNA for Adjuvant Effects

To compare a DNA form of an adjuvant vaccine, known to continuously induce protein expression, and a protein form of the adjuvant, encapsulated in microspheres, for adjuvant effects, five-week old BALB/c CrSlc mice were subcutaneously immunized with HBsAg (Euvax B, LGCI Co. Ltd.) and the IL-12-encapsulating microspheres. After two weeks, the titers of total serum IgG, IgG1, and IgG2a antibodies were measured by an anti-S ELISA. Separately, five-week old BALB/c CrSlc mice were immunized with HBsAg by subcutaneous injection and IL-12 DNA (ACP30-mIL-12, POSTECH Cellular Immunology Lab.) by intramuscular injection, and, after two weeks, the titers of total serum IgG, IgG1, and IgG2a antibodies were measured by an anti-S ELISA. As shown in FIGS. 5a to 5c, a rIL-12-encapsulating microsphere-administered group (Group 3) was found to induce stronger HBsAg-specific total IgG, IgG1, and IgG2a antibody responses than an IL-12 DNA-administered group (Group 2). These results indicate that the rIL-12-encapsulating microspheres of the present invention are superior as an adjuvant to the IL-12 DNA known to induce sustained expression of a gene encoding IL-12.

### EXAMPLE 6

### Enhanced Influenza HA-specific Antibody Responses by the rIL-12-Encapsulating Microspheres

To investigate the adjuvant effect of the rIL-12-encapsulating microspheres with respect to antibody responses, five-week old BALB/c CrSlc mice were intranasally immunized twice at intervals of two weeks with an influenza HA protein (Influenza HA vaccine, LG Household & Health Care Co. Ltd.) and the microspheres prepared in Example 1, which both were suspended in a suspension solution (3% carboxymethyl celluose, 8.7 mg/ml NaCl, 0.1% Tween 20). After eight weeks, the titers of total serum IgG, IgG1, and IgG2a antibodies were measured by an anti-HA ELISA to determine whether antigen-specific antibody responses had been induced. FIG. 6 shows the results of the quantitative comparison of test groups for antibody responses by an end-point dilution assay. As shown in FIG. 6, Group 2, administered with the antigen and rIL-12, induced almost identical antibody responses to Group 4 administered with one-fifth of the amount of the rIL-12-encapsulating microspheres used in Group 2. In contrast, in Group 3 administered with the rIL-12-encapsulating microspheres in the same amount as in Group 2, total serum IgG, IgG1 and IgG2a antibody responses were significantly increased. In particular, with respect to IgG2a responses, Group 3, administered with the rIL-12-encapsulating microspheres, induced much stronger antibody responses than other groups.

These results indicate that the rIL-12-encapsulating microspheres effectively increase antigen-specific antibody responses and Th1 immune responses and are applicable diverse antigens other than HBsAg.

In addition, when Group 3 and Group 4, immunized with different amounts of the rIL-12-encapsulating microspheres, were compared with each other, antibody responses were increased along with the administered amount of the microspheres.

### EXAMPLE 7

### Enhanced Influenza HA-specific CTL Responses by the rIL-12-Encapsulating Microspheres

To investigate the adjuvant effect of the rIL-12-encapsulating microspheres with respect to CTL responses, five-week old BALB/c CrSlc mice were intranasally immunized twice at intervals of two weeks with an influenza HA protein (Influenza HA vaccine, LG Household & Health Care Co. Ltd.) and the microspheres prepared in Example 1, which both were suspended in a suspension solution (3% carboxymethyl celluose, 8.7 mg/ml NaCl, 0.1% Tween 20). After 11 weeks, virus infection was carried out with an influenza virus. Five days after the virus infection, lungs were excised from the mice, and lung lymphocytes were isolated by a Lympho-prep technique. CD8⁺ T cells in the lung were isolated, stimulated with an influenza HA-specific CLT epitope, and stained with CD8+ and IFN-γ-spcific antibodies. IFN-γ-secreting HA-specific CD8+ T cell levels were analyzed by FACS.

As shown in FIGS. 7a to 7d, Group 2, administered with rIL-12, had no significant difference with Group 1 in CTL responses. In contrast, Group 3, administered with the rIL-12-encapsulating microspheres, induced much stronger CTL responses than other groups.

With respect to immune responses by memory T cells produced after immunization of mice, these results indicate that the rIL-12-encapsulating microspheres are effective in enhancing immune responses by antigen-specific memory T cells.

### EXAMPLE 8

### Improved Protection of Immunized Mice Against Influenza Challenge by the rIL-12-Encapsulating Microspheres

To determine whether enhanced antibody and CTL responses by rIL-12 encapsulated microsphere is correlated with in vivo protection against homologous influenza challenge, five-week old BALB/c CrSlc mice were intranasally immunized twice at intervals of two weeks with an influenza HA protein (Influenza HA vaccine, LG Household & Health Care Co. Ltd.) and the microspheres, which both were suspended in a suspension solution (3% carboxymethyl celluose, 8.7 mg/ml NaCl, 0.1% Tween 20). After 11 weeks, the vaccinated mice were challenged with lethal doses of influenza virus. As shown in FIG. 8, in which mice were compared between test groups for survival rate for nine days after the virus challenge, Group 2 administered with rIL-12 displayed a slightly increased viability of about 10%, which was not significant, in comparison with a control group, Group 1, not administered with the adjuvant. In contrast, Group 3, administered with the rIL-12-encapsulating microspheres, exhibited a significantly increased viability of about 65%.

These results indicate that the rIL-12-encapsulating microspheres also effectively increase host's protection against infectious diseases by significantly increasing antigen-specific antibody responses and CTL responses.

### INDUSTRIAL APPLICABILITY

As described hereinbefore, the present invention provides a vaccine composition comprising a pathogenic antigen and an IL-12 adjuvant encapsulated in sustained release microspheres. IL-12, as an adjuvant in the vaccine composition, is released in vivo for a prolonged period of time by being encapsulated in sustained release microspheres, thereby maximizing its adjuvant effect.

1. A vaccine composition for enhancing an adjuvant effect of IL-12 comprising a pathogenic antigen and an IL-12 adjuvant encapsulated in controlled release microspheres.

2. The vaccine composition as set forth in claim 1, wherein the pathogenic antigen is selected from the group consisting of viruses, bacteria, parasites and fungi.

3. The vaccine composition as set forth in claim 2, wherein the pathogenic antigen is selected from the group consisting of hepatitis B virus, hepatitis C virus, human immunodeficiency virus, influenza virus and mycobacteria.

4. The vaccine composition as set forth in claim 1, wherein the pathogenic antigen is in a protein or peptide form.

5. The vaccine composition as set forth in claim 1, wherein the IL-12 is a recombinant IL-12.

6. The vaccine composition as set forth in claim 1, wherein the controlled release microspheres are manufactured by double emulsion-solvent evaporation.

7. A method of enhancing an adjuvant effect of IL-12, which is characterized by employing IL-12 encapsulated in controlled release microspheres as an adjuvant in a vaccine composition comprising a pathogenic antigen.

8. The method as set forth in claim 7, wherein the vaccine composition is administered subcutaneously or intranasally.
